(19) United States
(12) Patent Application Publication
(10) Pub. No.: US 2007/0088011 A1
(43) Pub. Date: Apr. 19, 2007

(54) Combination preparation for oral contraception and oral therapy of dysfunctional uterine bleeding containing estradiol valerate and dienogest and method of using same

(76) Inventors: Susan Zeun, Berlin (DE); Pol Boudes, Hackettstown, NJ (US); Angelo Secci, Parsippany, NJ (US); Jan Endrikat, Kirkland (CA); Holger Zimmermann, Falkensee (DE)

Correspondence Address: STRIKER, STRIKER & STENBY, 103 EAST NECK ROAD, HUNTINGTON, NY, 11743, US

(21) Appl. No.: 11/377,693

(22) Filed: Mar. 16, 2006

Related U.S. Application Data
(60) Provisional application No. 60/727,592, filed on Oct. 17, 2005.

(51) Int. Cl. A61K 31/57; A61K 31/56

(52) U.S. Cl. 514/170

(57) ABSTRACT

The multiphase combination preparation for oral therapy of dysfunctional uterine bleeding and for oral contraception contains a first phase of 2 daily dosage units, each consisting of 3 mg of estradiol valerate; a second phase of two groups of daily dosage units, which consist of a first group containing 5 daily dosage units, each of which consist of a combination of 2 mg of estradiol valerate and 2 mg of dienogest, and a second group containing 17 daily dosage units, each of which consist of a combination of 2 mg of estradiol valerate and 3 mg of dienogest; a third phase of 2 daily dosage units each consisting of 1 mg of estradiol valerate; and another phase of 2 daily dosage units of a pharmaceutically harmless placebo; so that the multiphase combination preparation consists of a total number of 28 daily dosage units.

### CROSS-REFERENCE

The invention disclosed here is the same as the invention disclosed in U.S. Provisional Application Ser. No. 60/727,592, filed Oct. 17, 2005 on which a claim of priority under 35 U.S.C. 119 for the present invention disclosed herein is based.

### BACKGROUND OF THE INVENTION

The subject matter of the present invention comprises the use of estradiol valerate in combination with 17α-cyanomethyl-17β-hydroxyestra4,9-dien-3-one (dienogest) containing a first phase of 2 daily dosage units of 3 mg of estradiol valerate; a second phase of two groups of daily dosage units, a first group of which containing 5 daily dosage units of a combination of 2 mg of estradiol valerate and 2 mg of dienogest and a second group of which containing 17 daily dosage units of a combination of 2 mg of estradiol valerate and 3 mg of dienogest; a third phase of 2 daily dosage units with 1 mg of estradiol valerate and another phase of 2 daily dosage units of pharmaceutically harmless placebo for preparing a multiphase combination preparation with a total number of 28 daily dosage units for oral therapy of dysfunctional uterine bleeding and for oral contraception.

The total number of daily dose units of the multiphase combination and the pharmaceutically harmless placebo is sufficient for 28 days.

### 2. DESCRIPTION OF THE RELATED ART

Dysfunctional uterine bleeding (DUB) is a frequent clinical problem in gynecology and affects up to 33% of women presenting themselves for gynecological medical examinations on an outpatient basis (Awward J. T., Toth T. L., Schiff I., Abnormal Uterine Bleeding in the Perimenopause, Int. J. Fertil. 1993; 38, pp. 261-9).

The symptoms of DUB are:

extended menstrual bleeding (>7 days)
frequent bleeding (interval between bleeding episodes of less than or equal to 21 days)
increased bleeding (more than or equal to 80 ml).

DUB requires a diagnosis by exclusion, namely organic causes such as myoma, polyps or cancer must be excluded before a DUB diagnosis can be made.

DUB is associated with anovulation as well as ovulation. Such bleeding disturbances are due to an imbalance between the estrogen-stimulating build-up phase (proliferation) of the endometrium and the gestagenic transformation of the endometrium. If the DUB symptoms are a result of chronic anovulation, the endometrium is often exposed to increased gestagenic proliferation. Such proliferation can lead to hyperplasia of the endometrium besides the bleeding disturbances (Speroff, et al., Clinical Gynecologic Endocrinology and Infertility, sixth edition, Lippincott, Williams and Wilkins, 1999).

Hyperplasia of the endometrium is a risk factor for the onset of endometrial cancer.

Fraser, I. S., Aust. N. Z. J. Obstet. Gynaecol. (1990) 30 (4), pp. 353-356, reported the treatment of dysfunctional uterine bleeding by administration of 5 mg of norethisterone, three times daily, or 10 mg of medroxyprogesterone acetate, three times daily, as the only high-dosage gestagen, in each case for 14 days from the 12^{th} to the 25^{th} day of the cycle in 6 anovulatory women and for 20 days from the 5″¹ to the 25^{th} day of the cycle in ten ovulatory women. In both groups, the duration of the bleeding period was reduced. Reliable contraception was not attained.

Hickey M., Higham J. and Fraser I. S, The Chochrane Library, Issue 3 2004 (Mickey M, Higham J, Fraser I S, Progestogens Versus Estrogens and Progestogens for Irregular Uterine Bleeding Associated with Anovulation (Cochrane Review). In The Cochrane Library, Issue 3 2004, Chichester, UK: John Wiley & Sons, Ltd) describe in a review article the low tolerance of women for irregular and extensive bleeding. They describe the rationale behind the use of gestagens to achieve a transformation of the endometrium and thus to create more stable menstruation cycles. The conclusion of the article is that clinical data from randomized studies demonstrating the efficacy of the described treatments are currently not available.

Steiner, R., Schweiz. Rundsch. Med. Prax. (2000) 91 (46), pp. 1967-1974, also points out that dysfunctional uterine bleeding should be treated with, among other methods, high-dosage gestagens, estrogens or a combination of both.

Steiner sees a treatment regimen in the oral administration of 0.01 mg of ethinyl estradiol with 2 mg of norethisterone acetate for 8 days in decreasing dosages, namely 6, 5, 4, 3, 3, 3, 3, 3/day. Besides the hormonal approach, Steiner postulates the possibility of treating an acute bleeding situation with tranexaminic acid, up to 4×2 tablets per day.

Davis, A., Obstet. Gynecol. (2000) 96 (6), pp. 913-920, describes the treatment of dysfunctional uterine bleedings by a three-step administration of ethinyl estradiol (EE)/norgestimate (NGM) followed by hormone-free administration of placebo for three 28-day cycles. According to the treatment regimen, the EE dosage remains constant over 21 days (0.035 mg of EE), the NGM dose increases over 21 days (7 daily dosage units of 0.180 mg of NMG and 7 daily dosage units of 0.215 mg of NMG and 7 daily dosage units of 0.250 mg of NMG), followed by a 7-day hormone-free placebo administration. The placebo-controlled study carried out by Davis included 45% of women with increased menstrual bleeding (metrorrhagia, menometrorrhagia and polymenorrhea) and about 55% of women with reduced menstrual bleeding (oligomenorrhea). The highest degree of success compared to placebo was achieved in women with reduced menstrual bleeding in whom regular withdrawal bleeding was induced. Oligomenorrhea is not necessarily a component of the DUB symptom group and is not recognized as an ailment worthy of treatment.

U.S. Pat. No. 6,782,282 states generally that extended use (3 months) of oral contraceptives can be used for the treatment of menorrhagia—a form of dysfunctional uterine bleeding.

### SUMMARY OF THE INVENTION

The object of the invention is to develop means for the treatment of dysfunctional uterine bleeding that will generally reduce the extent of bleeding and will prevent the recurrence of dysfunctional bleeding, while at the same time ensuring reliable, safe and well-tolerated oral contraception.

By the term “dysfunctional uterine bleeding” is meant here extended menstrual bleeding lasting more than 7 days with an interval between bleeding episodes of less than or equal to 21 days, or increased bleeding of more than or equal to 80 ml without an organic cause.

According to the invention this objective is attained by a multiphase combination preparation for oral therapy of dysfunctional uterine bleeding and for oral contraception, which is based on estradiol valerate in combination with 17α-cyano-methyl-17β-hydroxyestra-4,9-dien-3-one (dienogest). The multiphase combination preparation contains a first phase of 2 daily dosage units consisting of 3 mg of estradiol valerate; a second phase of 2 groups of daily dosage units, including a first group containing 5 daily dosage units of a combination of 2 mg of estradiol valerate and 2 mg of dienogest and a second group containing 17 daily dosage units of a combination of 2 mg of estradiol valerate and 3 mg of dienogest; a third phase of 2 daily dosage units with 1 mg of estradiol valerate and another phase of 2 daily dosage units of a pharmaceutically harmless placebo.

The total number of daily dosage units of the multiphase combination preparation and the pharmaceutically harmless placebo is sufficient for 28 days.

The duration of use comprises at least one treatment cycle and depends on the individual desires of the woman regarding contraception.

### Studies of the Efficacy of the Claimed Formulation

180 women 18 to 50 years of age with DUB symptoms, in whom an organic cause of the symptoms had been excluded by appropriate diagnostic methods (transvaginal ultrasound, hormone determination in the blood) and who had given their written consent to participate in the study, were treated in a randomized, double-blind, placebo-controlled clinical study. 120 women received estradiol valerate and dienogest in accordance with the claimed combination and 60 women received placebo.

The study comprised a run-in phase of 90 days during which the severity of the bleeding disturbances was recorded, 6 treatment cycles and one post-treatment cycle (follow-up phase).

The extent of bleeding was determined quantitatively by the alkaline hematin method. To this end, the women collected the monthly discharges during the entire study period and gave them to the testing center. The duration of the bleeding and the duration of the bleeding-free intervals were recorded by daily documentation in an electronic journal.

While the invention has been illustrated and described as embodied in a combination preparation for oral contraception and oral therapy of dysfunctional uterine bleeding containing estradiol valerate and dienogest and method of using same, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A multiphase combination preparation for oral contraception and oral therapy of dysfunctional uterine bleeding, said combination preparation containing
a first phase of two daily dosage units, each of which consist of 3 mg of estradiol valerate;
a second phase of two groups of daily dosage units, said two groups consisting of a first group containing 5 daily dosage units, each of which consist of a combination of 2 mg of estradiol valerate and 2 mg of dienogest, and a second group containing 17 daily dosage units, each of which consist of a combination of 2 mg of estradiol valerate and 3 mg of dienogest;
a third phase of two daily dosage units, each consisting of 1 mg of estradiol valerate; and
another phase of two daily dosage units, each consisting of a pharmaceutically harmless placebo;
so that the multiphase combination preparation consists of a total number of 28 daily dosage units.

2. A method of treating dysfunctional uterine bleeding and of oral contraception, said method comprising the steps of:
a) providing a multiphase combination preparation for oral contraception and oral therapy of dysfunctional uterine bleeding, said combination preparation containing a first phase of two daily dosage units each consisting of 3 mg of estradiol valerate; a second phase of two groups of daily dosage units, said two groups consisting of a first group containing 5 daily dosage units, each of which consist of a combination of 2 mg of estradiol valerate and 2 mg of dienogest, and a second group containing 17 daily dosage units, each of which consist of a combination of 2 mg of estradiol valerate and 3 mg of dienogest; a third phase of 2 daily dosage units with 1 mg of estradiol valerate; and another phase of 2 daily dosage units of a pharmaceutically harmless placebo; so that the multiphase combination preparation consists of a total number of 28 daily dosage units; and
b) administering said multiphase combination preparation to a woman in need of treatment for dysfunctional uterine bleeding over at least one treatment cycle.

3. The method as defined in claim 2, wherein said at least one treatment cycle consists of six treatment cycles.
